SEQUENCE LISTING (2 sequences)

SEQ ID NO: 1 - protein, 321 aa, Gallus gallus
MAKYTRGTVTAFSPFDARADAEALRKAMKGMGTDEETILKILTSRNNAQRQEIASAFKTLFGRDLVDDLKSELTGKFETLMVSLMRPARIFDAHALKHAIKGAGTNEKVLTEILASRTPAEVQNIKQVYMQEYEANLEDKITGETSGHFQRLLVVLLQANRDPDGRVDEALVEKDAQVLFRAGELKWGTDEETFITILGTRSVSHLRRVFDKYMTISGFQIEETIDRETSGDLEKLLLAVVKCIRSVPAYFAETLYYSMKGAGTDDDTLIRVMVSRSEIDLLDIRHEFRKNFAKSLYQMIQKDTSGDYRKALLLLCGGDDE

SEQ ID NO: 2 - protein, 313 aa, Gallus gallus
MAAFSPFDARADAEALRKAMKGMGTDEETILKILTSRNNAQRQEIASAFKTLFGRDLVDDLKSELTGKFETLMVSLMRPARIFDAHALKHAIKGAGTNEKVLTEILASRTPAEVQNIKQVYMQEYEANLEDKITGETSGHFQRLLVVLLQANRDPDGRVDEALVEKDAQVLFRAGELKWGTDEETFITILGTRSVSHLRRVFDKYMTISGFQIEETIDRETSGDLEKLLLAVVKCIRSVPAYFAETLYYSMKGAGTDDDTLIRVMVSRSEIDLLDIRHEFRKNFAKSLYQMIQKDTSGDYRKALLLLCGGDDE

(19) United States
(12) Patent Application Publication
(10) Pub. No.: US 2005/0059587 A1
(43) Pub. Date: Mar. 17, 2005

(54) Use of a protein for the production of a medicament for stimulating the innate non-specific immune system

(76) Inventors: Martin Herrmann, Neunkirchen (DE); Wolf Bertling, Erlangen (DE); Joachim R. Kalden, Erlangen (DE); Reinhard E. Voll, Eggolsheim (DE); Angelo Manfredi, Mailan (IT); Wasilis Kolowos, Schondra (DE); Christian Reiser, Bamberg (DE); Walter Woith, Erlangen (DE)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/489,373

(22) PCT Filed: Aug. 24, 2002
(86) PCT No.: PCT/EP02/09473

(30) Foreign Application Priority Data
Sep. 13, 2001 (DE) 101-45-254.3

(51) Int. Cl. A61K 38/17

(52) U.S. Cl. 514/12

(57) ABSTRACT

The invention relates to the use of a protein for producing a medicament for stimulating an innate non specific immune system, wherein the protein contains a) an annexine V or a largely similar molecule thereto or b) an active fragment of annexine V or a largely similar molecule thereto.

The invention relates to a use of a protein for the production of a medicament and to a medicament for stimulating the innate non-specific immune system. It also relates to a method for manufacturing the medicament.

The pathological cell death, necrosis, is connected with a swelling of the cell organelles and the cell itself. The cytoplasma membrane becomes permeable and the contents of the cell are released. In contrast to this, the cell membrane remains intact at first during the programmed cell death, apoptosis. The cells are removed before their possible dangerous content is released to the environment. During an early apoptosis stage the cells are subject to surface changes, for example, a modification of the carbohydrates and the exposure of anionic phospholipides, in particular phosphatidylserine (PS) on the outside of the cytoplasma membrane. The latter is caused by the downward adjustment of the ATP-dependent aminophospholipid-translocasis which specifically transports aminophospholipides from the outside to the inside of the membrane. In addition, a non-specific lipid flip site known as scramblase is activated which causes an acceleration of the phosphatidylserine flip-flop mechanism. Phosphatidylserine located on the outside of the cytoplasma membrane serves as identifying signal for the removal of apoptotic cells.

It is known that the pre-incubation of apoptotic lymphocytes with annexin V (AxV) effectively blocks the absorption of apoptotic cells by murine peritoneal macrophages, macrophages of the J774 cell line of the mouse and bone marrow macrophages (Krahling, S., et al., Cell Death Differ. 6 (1999), 183-189). In addition, annexin V generally causes stronger inhibition of the absorption of apoptotic cells both by activated and non activated macrophages. Due to its Ca²⁺ canal activity, annexin V also slows the apoptosis in CEM cells.

The removal of apoptotic cells normally induces neither an inflammation nor an immune response. In direct contact with plasma, apoptotic cells can cause pro-coagulating and, under certain circumstances, pro-inflammatory effects. However, immune-modulating effects of apoptotic cells normally dominate on monocytes/macrophages through the interaction of apoptotic cells via thrombospondin with CD36 on phagocytes.

Lektin-like molecules such as the vitronectin receptor (CD51/CD61), thrombospondin, CD36 and CD14 are receptors which recognize surface changes on apoptotic cells. CD14 appears to be necessary for the phagocytes of lymphocytes and lipid-symmetric erythrocytes with activated and non activated macrophages.

The phagocytosis of apoptotic lymphocytes due to macrophages is stereo-specifically inhibited by phosphatidyl-L-serine-liposoms. A receptor responsible for the recognition of the phosphatidyl-L-serine is defined by antibodies which were produced by the macrophages stimulated by the immunization with TGF-β and β-glucan (Fadok, V. A, et al., Nature 405 (6782) (2000), 85-90). Other important receptors for this recognition of apoptotic cells are, among others, the scavenger receptors, the LPS receptor CD14, the thrombospondin receptor CD36, the vitronectin receptor CD51/CD61, the complement receptors and the macrosialin CD68. Furthermore, pentraxine, collectine, different complement factors, β2-glycoprotein, the annexines and gas-6 can serve as adaptor proteins.

It is also known that mice which were injected with apoptotic human T cells showed significantly reduced humoral responses to T cells in comparison to control mice which were injected with viable cells (Ponner, B. B., et al., Scand. J. Immunol. 47 (1998), 343-347). It was also observed that an immunization with apoptotic cancer cells induced drastically reduced cytotoxic T cell responses in comparison to living, growth-blocking cells (Ronchetti, A., et al., J. Immunol. 163 (1999), 130-136). This shows that the engulfment phagocytosis of apoptotic cells does not lead to an efficient antigen presentation and activation of T and B lymphocytes. It is possible that a fast engulfment phagocytosis of apoptotic cells due to macrophages and their anti-inflammatory response prevents the absorption and efficient presentation of antigens stemming from apoptotic cells by dendritic cells. Together with a production of interleukin-10 (IL-10) by monocytes/macrophages after contact with apoptotic cells, these findings explain the poor efficiency of cancer vaccines containing apoptotic cells.

From Stach C. M. et al., Cell Death and Differentiation (2000) 7, 911-915 it is known that annexin V stimulates the humoral immune response. This article contains no information on a stimulation of the cellular immune response or the innate unspecific immune system.

The object of the invention is to remove the disadvantages in accordance with the state of art. In particular it is to be specified a use and a medicament against tumors, viruses, bacteria and parasites. Moreover, a method is to be specified for the manufacturing of the medicament.

This object is solved by the features of claims 1, 14 and 22. Useful arrangements of the inventions result from the features of claims 2 to 13, 15 to 21 and 23 to 33.

According to the invention, the use of a protein for the production of a medicament for stimulating of the innate non-specific immune system is provided, wherein the protein includes
a) annexin V or a molecule which is largely similar to it, or
b) an effective fragment of annexin V or the molecule which is largely similar to it.

Surprisingly it was shown that the use of the protein provided by the invention causes a pro-inflammatory response of the monocytes or macrophages and thus an increase in the cellular immunity. The used protein is excellently suited to combating tumors, viruses, bacteria and parasites.

The effect of the protein provided by the invention is evidently based on the apoptotic cells' being changed by a loading with annexin V so that they are able to activate the innate unspecific immune system, in particular natural killer cells (NK cells) and macrophages. Moreover the administration of annexin V increases the effective concentration of monokin TNK-α. Monokin TNK-α is particularly important for the recruiting of NK cells in the peritoneum (Smyth, M. J. et al., 1998, J. Exp. Med. 9:1611-1619). Moreover the effect of the protein provided by the invention is also particularly based on the fact that it binds to phosphatidylserine and its anti-inflammatory power. This function of the protein is not only fulfilled when annexin V is involved. It is also fulfilled when the protein only includes a molecule similar to annexin V or an effective fragment of annexin V or a molecule which is largely similar to the fragment.—A fragment is particularly “effective” when it binds to phosphatidylserine and contributes to the stimulation of a pro-inflammatory cellular immune response. The term “similar molecules” means such molecules which bind particularly to phosphatidylserine and have a certain degree of identity with annexin V.

In accordance with an embodiment an amino acid sequence of the protein can correspond to the amino acid sequence of SEQ ID no. 1 or no. 2 or be identical to it at least 50%, preferably at least 60%, particularly preferably at least 70%, very particularly preferably at least 80% of the time. The determination of the identity can be performed, for example, in accordance with the method of Altschul, S. F. et al. (1997), Nucleic Acids Res. 25:3389-3402.

The term “identity” means in this case the extent to which two nucleic or amino acid sequences are invariant.

With the amino acid sequence of SEQ ID no. 2, this is an N terminal deletion mutant of the amino acid sequence of SEQ ID no. 1 which is missing the eight amino acids 3 to 10, this means the amino acids Lys Tyr Thr Arg Gly Thr Val Thr.

It is useful that annexin V is non-human annexin V, preferably chicken annexin V. A comparison of the amino acid sequence of chicken annexin V with human annexin V shows that both proteins are 78.2% identical. The chicken annexin V has a theoretical iso-electrical point (pI) of 5.60 while human annexin V has a theoretical iso-electrical point of 4.94. The sequence of the human annexin can be called up under the access number P08756 in the “SWISS-PROT” database. Both annexins are immunogen in the animal model.

The stimulation of the immune response can be caused by a blockade, covering, masking and/or removal of extra-cellular, membrane-based, localized phosphatidylserine. The protein can thus be usefully used to combat bacteria, viruses, parasites or tumors. Preferably the protein is used as an auxiliary agent. In this connection it can preferably be used in tumor therapy, for tumor vaccines, in virus therapy, in particular for the treatment of retrovirus infections, lentivirus infections, infections with treponema pallidum, the sindbisvirus, trypanosoma brucei and HIV infections and for the treatment of malaria as well as for malaria immunization.

In addition to the substance, the medicament can also include human tumor cells, wherein the tumor cells can be apoptotic and/or necrotic tumor cells. The apoptosis and/or necrosis of the tumor cells can occur spontaneously or can have been induced. As inductors for the apoptosis and/or necrosis can be considered radiation of the tumor cells ex-vivo or in-vivo or bringing the tumor cells in contact with cytostatic medicaments. In this connection chemicals such as H₂O₂ or staurosporin, medicaments such as adrenocortical steriods, chemotherapeutic agents such as doxorubicin, cis-platin or hydroxy-urea, UVB and UVC radiation, as well as β, γ or X rays are particularly suitable. Preferably the apoptotic and/or necrotic tumor cells are brought into contact with the substance.

Further in accordance with the invention a medicament for stimulating of the innate non-specific immune system with a protein is provided, which includes
a1) annexin V or a molecule which is largely similar or
a2) an effective fragment of annexin V or of the molecule which is largely similar and
bb) apoptotic and/or necrotic tumor cells.

For the advantageous embodiments, reference is made to the preceding description. The features mentioned therein can also correspondingly be embodiments of the medicament.

Further in accordance with the invention a method for the production of the medicament provided by the invention is provided, wherein apoptotic and/or necrotic tumor cells are brought into contact with a protein which includes
a) annexin V or a molecule which is largely similar to it, or
b) an effective fragment of annexin V or the molecule which is largely similar to it.

For the advantageous embodiments, reference is then made to the preceding descriptions. The features specified there can also be used for embodiments of the method.

The protein used in this method is preferably made by expression via transformed strains of Escherichia coli and then purification. Preferably the strain Escherichia coli BL21 (DE3) is used. The used strain of Escherichia coli can be transformed with an expression plasmid which is capable of expressing chicken annexin V or an effective fragment thereof. For example, the expression plasmid is pDJ2-AnXV which contains in addition to the gen expressing chicken annexin V an IPTG-inducible tac promoter and an antibiotic-resistant cassette. Canamycin is preferably used as the antibiotic to separate the non transformed bacteria from the transformed bacteria.

The cells obtained by using the transformed strains of Escherichia coli which contain chicken annexin V after a culture in a culture medium are broken down in a buffer. The thus obtained solution which contains the chicken annexin V is purified with column chromatography, wherein a salt gradient, a sodium chloride gradient has been shown to be useful, is used. The substance is obtained with a purity which exceeds 95%.

The suitability of chicken annexin V or an effective fragment thereto as the substance is based on the determination that the masking of phosphatidylserine with this substance causes a pro-inflammatory effect of apoptotic and/or necrotic cells and thus supports the cellular immune response to apoptotic cells. Chicken annexin V significantly increases the immunogenicity of autologous or syngenous apoptotic cells. It is thus suitable for increasing the efficiency of vaccines containing apoptotic cells. This is then particularly important when the cells were radiated before an injection of the vaccines. Without the use of the substance chicken annexin V the immunogenicity of these cells is low since the absorption of apoptotic cells causes an anti-inflammatory reaction of human monocytes/macrophages. When the cells are treated with chicken annexin V, this anti-inflammatory effect of apoptotic and/or necrotic cells is reduced and their immunogenicity is significantly increased.

The protein provided by the invention can be used to block the anti-inflammatory effect of phosphatidylserine-exposing cells. Furthermore the protein can cause an immune stimulation via blockage, covering, masking and/or removal of extracellular, preferably membrane-based localized phosphatidylserine. The protein can thus be used to modulate a cellular immune response.

An important area of use for the blockage of the anti-inflammatory effects of phosphatidylserine-exposing cells is the specific “auxiliary agent effect” resulting from this. After the blockage of the anti-inflammatory principle of function, cells carrying phosphatidylserine are processed via a pro-inflammatory, immune-stimulating alternate route which causes a massively increased immune response. This “auxiliary agent effect” can be used for the most diverse of application areas, for example, in human medicine. On the one hand, the immunogenicity of tumor vaccines can be increased by this when these consist of radiated and thus largely apoptotic tumor cells. Furthermore it is possible to obtain a cellular immune response to such tumor cells which for therapeutic reasons have been treated with cytostatic medicaments or have received in situ radioactive radiation. In this case a tumor-specific cellular immune response would increase the success of the therapy during removal of the remaining tumor mass. Also with the treatment of virus infections, for example, covered viruses which expose phosphatidylserine, the blockage of the phosphatidylserine-dependent anti-inflammatory effect causes a specific immune stimulation. The treatment of infections with lentiviruses and HIV must be seen as a particularly important example in this connection. A phosphatidylserine-dependent invasion of the viruses which goes “unnoticed” by the cell causes virus persistence in the long-living monocyte/macrophage pool. The virus persistence leads to death in almost all those infected after a more or less long period of latency. Chicken annexin V is suitable for the treatment of those who are HIV-infected since inflammatory, phagocytising, apoptotic material triggers a “respiratory burst” in the phagocytes and thus destroys the virus genomes.

This similarly also applies to the treatment of infections with phosphatidylserine-exposing bacteria (e.g., treponema pallidum) or of infections and parasitic illnesses which cause the exposure of phosphatidylserine on infected host cells (e.g., sindbisvirus, plasmodium falciparum, trypanosoma brucei).

The invention will now be described in more detail based on the drawings. The figures are listed below:

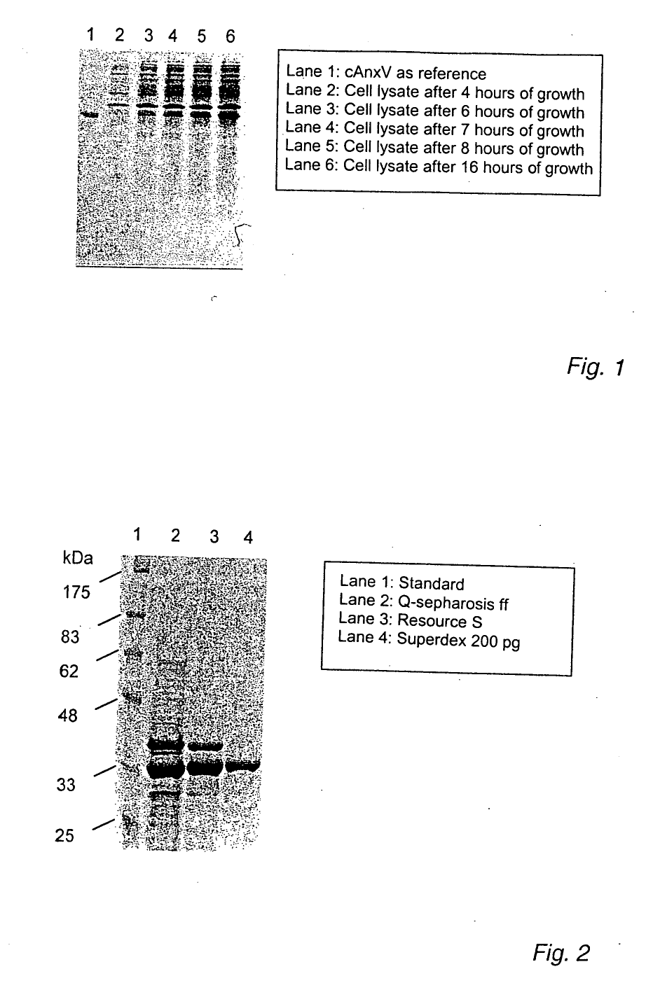

FIG. 1 The expression kinetics of chicken annexin V in transformed Escherichia Coli BL21 (BE3) based on a polyacrylamide-gel-electrophoresis,

FIG. 2 A polyacrylamide-gel-electrophoresis of samples of the individual purification steps of chicken annexin V from transformed Escherichia Coli BL21 (DE3),

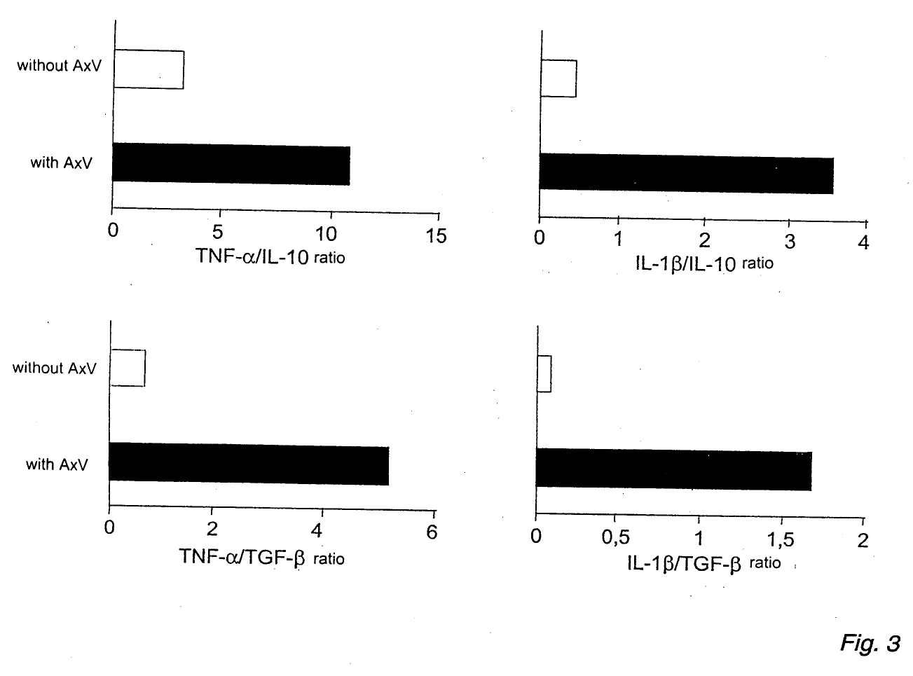

FIG. 3 Relationships of secreted cytokine in dependence on the incubation of murine peritoneal macrophages with radioactive radiated tumor cells which were treated before with chicken annexin V or not with annexin V,

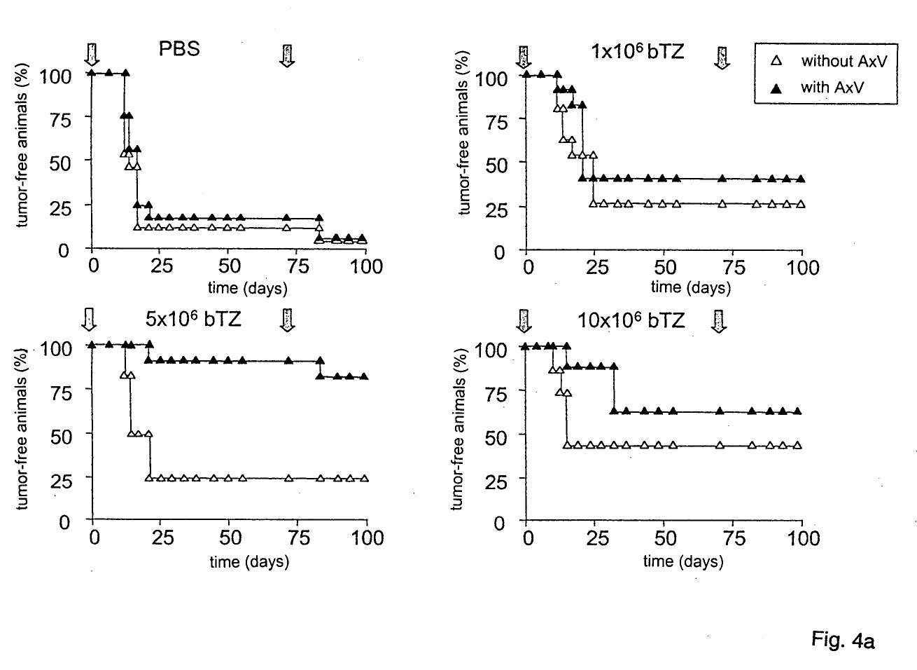

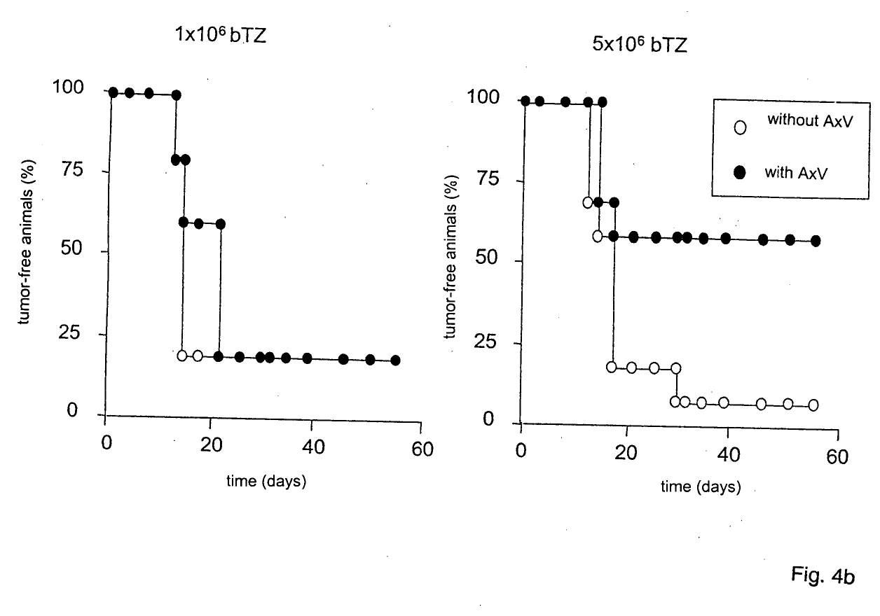

FIG. 4a The portion of tumor-free animals in dependence on the time and the injection of different amounts of radiated tumor cells which were treated after the radiation with or without chicken annexin V, wherein the animals were treated with living tumor cells before the injection,

FIG. 4b The portion of tumor-free animals in dependence on the time and the injection of radiated tumor cells which were treated after the radiation with or without chicken annexin V, wherein the animals were injected with living tumor cells before the treatment,

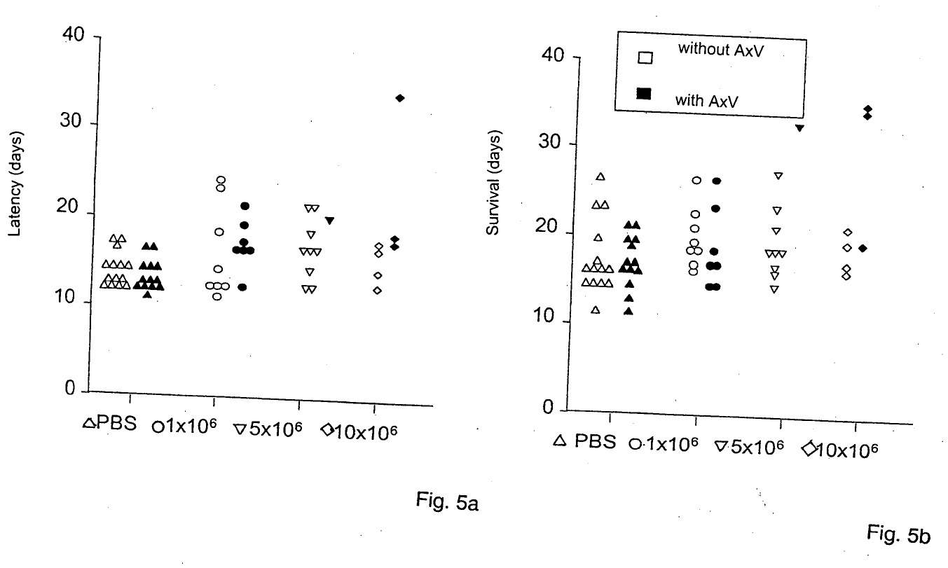

FIG. 5a Tumor latency of the mice which did not reject the tumor in dependence on the dose of radiated tumor cells and the administration of annexin V and

FIG. 5b Survival of mice which did not reject the tumor in dependence on the dose of radiated tumor cells and the administration of annexin V,

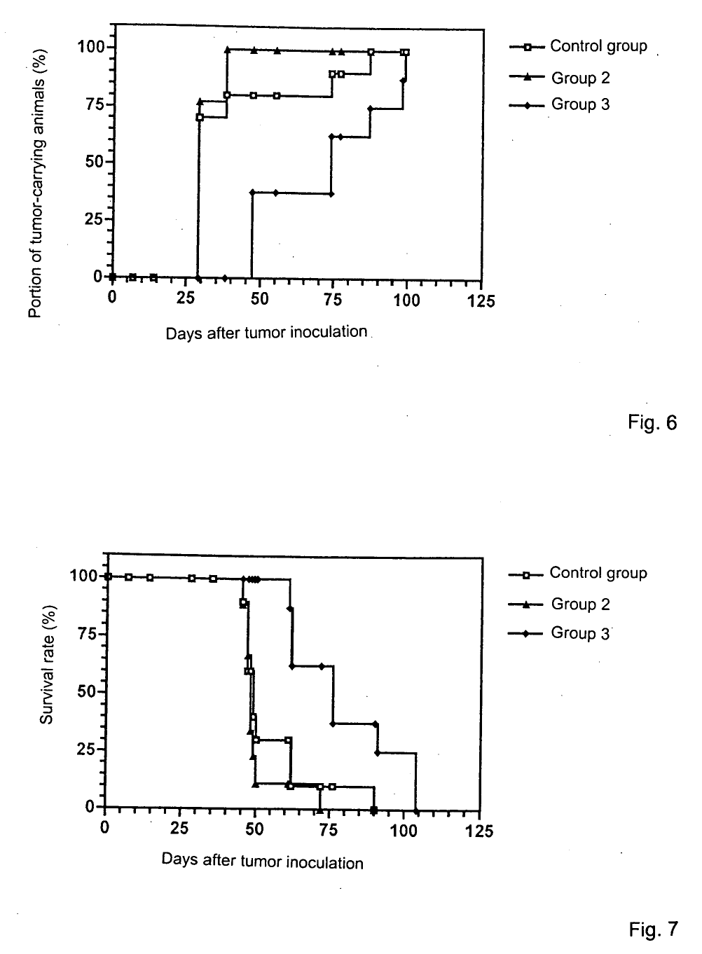

FIG. 6 The portion of tumor-carrying animals in dependence on the duration after the tumor inoculation,

FIG. 7 The survival rate in dependence on the duration after the tumor inoculation,

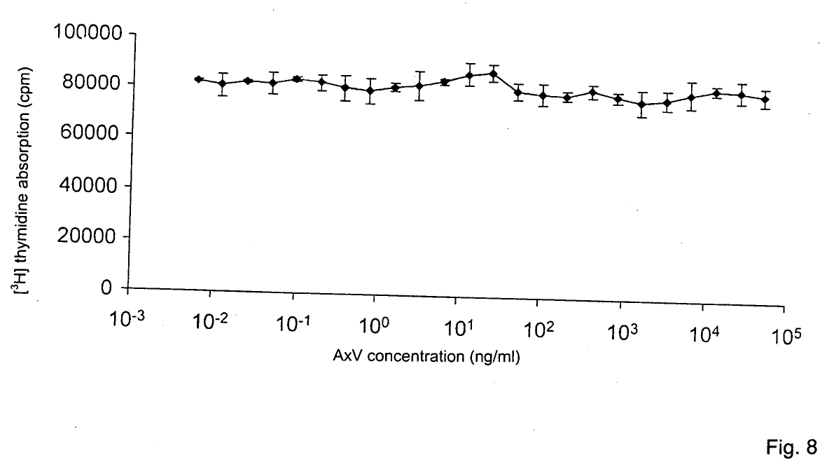

FIG. 8 The [³H]-thymidine absorption of INA6 TU1 cells via the AxV concentration and

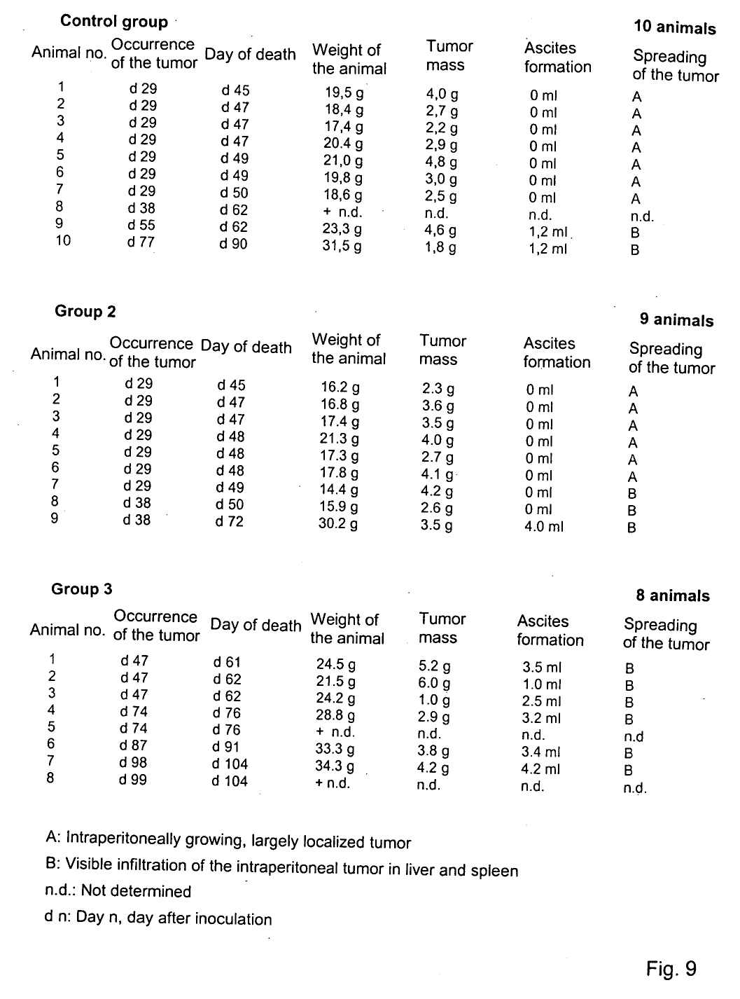

FIG. 9 Summarizing table of the results of the examined animal groups.

### A. EXPRESSION AND PURIFICATION OF CHICKEN ANNEXIN V

### A1. EXPRESSION OF CHICKEN ANNEXIN V

For the expression of chicken annexin V (cAnxV) pDJ2-AnxV was used as the expression plasmid which, in addition to the cAnxV coding gene, contains an IPTG-inducible tac promoter and a canamycin-resistance cassette. E. coli BL21 (DE3) was transformed with the expression plasmid and expression kinetics were performed. 2xYT with 50 mg/1 canamycin was used as the culture medium for this.

To manufacture cAnxV a 100-ml preliminary culture was injected with freshly transformed E. coli BL21 (DE3) and shook at 37° C. for 8 hours. 5 l of the principal culture were mixed with 5 ml of the preliminary culture and shook at 37° C. for 16 to 20 hours. IPTG was not added since, in this case, the same expression yields were obtained with and without induction. The cells were then harvested by centrifugation. The cell wet mass was 16 to 21 g. The expression kinetic is shown in FIG. 1. The expression kinetics showed that E. coli BL21 (DE3) is also suitable for an expression of cAnxV in the used medium without induction with IPTG.

### A2. PURIFICATION OF CHICKEN ANNEXIN V

The cells obtained in accordance with number 1 were resuspended in a buffer A1 (20 mM Tris/CL1 pH 7.5, 2 mM EDTA) and broken down via high pressure (gaulin). Insoluble components of the break-down suspension were removed via high-speed centrifugation. The soluble supernatant containing cAnxV was applied to a Q-sepharosis-ff column(column 1) equilibrated in buffer A1 (25 ml, Amersham Pharmacia, Freiburg). The elution of the object protein was performed with a linear NaCl gradient. The fractions containing cAnxV were combined and dialyzed through a buffer A2 (50 mM Na-acetate pH 5.6). The dialysate was applied to a resource S column (column 2) equilibrated in A2 (6 ml, Amersham Pharmacia, Freiburg) and cAnxV was eluted with a linear NaCl gradient. The united fractions containing cAnxV were concentrated via ultra-filtration (Pall Filtron, USA) and applied to a Superdex 200 pg column (column 2) equilibrated in a 10 mM Na phosphate pH 7.2, 140 mM NaCl (Amersham Pharmacia, Freiburg). Homogenous cAnxV was eluted from the column. From 20 g of cells (wet mass) 30 mg of cAnxV were isolated with a purity exceeding 95%. FIG. 2 shows the results of purification using columns 1 to 3 based on a polyacrylamid gel-electrophoresis.

As an alternative, instead of the specified columns, Q-sepharosis XL can be used for column 1 (Amersham Pharmacia, Freiburg), SP-sepharosis HP can be used for column 2 (Amersham Pharmacia, Freiburg) and/or sephacryl S200 HR can be used for column 3 (Amersham Pharmacia, Freiburg).

As an alternative, a hydrophobic column can be used instead of the size-exclusion-chromatography (SEC) via column 3. In this case, the fractions containing cAnxV which elute from column 2 are united and increased with solid ammonium sulphate to 1.5 M. The protein solution is applied to a phenylsepharosis ff-column (15 ml, Amersham Pharmacia, Freiburg) and cAnxV is eluted with a linear gradient of 1.5 to 0 M ammonium sulphate.

### B. IMMUNIZATION OF MICE WITH ANNEXIN V-TREATED, APOPTOTIC TUMOR CELLS

### B1. MICE AND TUMOR CELLS

Six to eight weeks old, female C57BL/6 mice were kept in a pathogen-free animal experiment facility in accordance with the guidelines of the European Community. The syngenous H-2b lymphoma line RMA was made available by Angelo A. Manfredi (Hospitale San Raffaele, 20132 Milan, Italy). The H-2b melanoma cell line B16F1 was obtained from the American Type Culture Collection (ATCC, Rockville, Md.). The cell lines were tested regularly for mycoplasma infection.

### B2. CELL DEATH

RMA cells were radiated with ultraviolet light as described by Bellone, M., et al., J. Immunol. 159 (1997), 5391-5399, and then treated for 1 hour at 37° C. with 50 μg/ml mitomycin C and washed. The externalization of phosphatidylserine was followed by coloration with FITC-marked annexin V (Bender MedSystems, Valter Occhiena, Torino, Italy). The ion density of the plasma membrane was determined with the aid of propidiumiodid. The cells were analyzed 0, 3, 9 and 18 hours after radiation. The specificity of the annexin V bond was determined by treatment of radiated cells with increasing concentrations (1500 mg/ml) of unmarked annexin V or bovine serum albumin (BSA) and then determining the remaining annexin V—fluorescein-isothio-cyanat bond via flow cytometry (FACScan, Becton-Dickerson, San Jose, USA).

### B3. MACROPHAGES

Macrophages were isolated by adhesion to plastic from inflammatory peritoneal lavages which were induced by injection of thioglycolat (Fadok, V. A., et al., Nature 405 (2000), 85-90).

### B4. DETERMINATION OF THE CYTOKINE RELEASE OF MACROPHAGES AFTER CO CULTURE WITH RADIATED TUMOR CELLS

Macrophages were co-cultivated under different conditions with radiated tumor cells. In the culture supernatant the cytokines TNF-alpha, IL-1beta, IL-10 and TGF-beta were measured with the help of specific commercial ELISAs (DuoSet ELISA, R&D Systems, Minneapolis, Minn.).

### B5. IMMUNIZATION AND CURE

Immunization of the mice was done by subcutaneous (s.c.) injection into the balls of the feet of 1, 5 or 10×10⁶ radiated RMA cells. Either without or with 100 μg/ml chicken annexin V, the radiated cells were pre-incubated for 20 minutes at room temperature in an isotonic buffer containing Ca²⁺. On day 15 the mice received a booster injection into the right side. On day 30 the effectiveness of the immunization was tested by s.c. injection of 2.5×10⁴ living RMA cells into the opposite side. Twice a week it was then checked by feeling to determine whether tumors were growing in the animals. An animal was considered tumor positive when the average value of the two diameters vertical to each other was greater than 2 mm. When a tumor grew >10 mm or ulcerated, the affected animals were killed. Animals for which no tumor could be felt within 10 weeks after the injection of living RMA cells were considered tumor negative. These tumor-free mice were then given another injection of 2.5×10⁴ RMA cells into the opposite side to test the duration of protection. The specificity of the anti-tumor effect was tested by s.c. administration of 2.5×10⁴ syngenous B16F1 melanoma cells (Bellone, M., et al., J. Immunol. 158 (1997), 783-789). To determine the effect of differences in the treatment of established RMA lymphomas, 2.5×10⁴ living RMA cells were injected s.c. into the left side. After the tumor had grown for three days, the animals were treated on the opposite side with 1 or 5×10⁶ radiated RMA cells without or with chicken annexin V (one vaccination per week over three weeks). Tumor growth was determined as described above.

### B6. RESULTS

Murine peritoneal macrophages were incubated with different amounts of radioactively radiated tumor cells (bTZ) (syngenous RMA tumor cells) which were previously treated with either chicken annexin V or not with chicken annexin V. After 24 hours the culture supernatant was harvested and the release of cytokines TNG-alpha, IL-1-beta, IL-10 and TGF-beta was measured with ELISAs. FIG. 3 shows the results of the trials with a bTZ/Mph ratio of 5 to 1. The black bars show the results of radiated tumor cells which were treated with chicken annexin V. The white bars show the results of radiated tumor cells which were not treated with chicken annexin V. In table 1 which follows the individual values of the measurements are summarized again.

TABLE 1

| bTZ: | 1:1 | | 5:1 | |
|---|---|---|---|---|
| Mph Ratio | bTZ | bTZ + AxV | bTZ | bTZ + AxV |
| TNF-alpha | 257 +/− 30 | 763 +/− 66* | 335 +/− 30 | 978 +/− 48** |
| IL-1-beta | n.d. | n.d. | 44 +/− 7 | 322 +/− 85* |
| IL-10 | 97 +/− 23 | 95 +/− 4 | 105 +/− 30 | 90 +/− 8 |
| TGF-beta | 339 +/− 39 | 145 +/− 12 | 505 +/− 54 | 193 +/− 26* |

*P < 0.05
**P < 0.01

Treatment of apoptotic cells with chicken annexin V shifts the cytokine pattern of phagocytes in the direction of inflammation.

Radiated syngenous tumor cells which were treated with chicken annexin V protect and cure mice which were injected with a lethal tumor dose. Treatment creates a long-term, specific anti-tumor immunity.

FIG. 4a and 4b each show the portion of tumor-free animals in dependence on the time and the injection of different amounts of radiated tumor cells (bTZ) which were incubated after radiation with or without chicken annexin V. The results shown with filled symbols in FIGS. 4a and b relate to radiated cells which were treated with chicken annexin V. The white symbols relate to the results with radiated cells which were treated without chicken annexin V.

FIG. 4a shows results of trials during which mice which were injected with living tumor cells (vaccination attempt) after the injection of radiated tumor cells which were incubated after radiation with or without chicken annexin V. The injection of living tumor cells was made on day 0 (1st arrow) and on day 72 (2nd arrow) for the surviving animals.

C57B1/6 mice were subcutaneously injected twice in the right side based on the following pattern.
With PBS
With 1×10⁶ radiated tumor cells (bTZ)
With 5×10⁶ radiated tumor cells (bTZ)
With 1×10⁷ radiated tumor cells (bTZ)

On day 0, two weeks after the last booster injection, 2.5×10⁴ living RMA tumor cells were injected into the opposite left side of the mice. On day 72 the injection was repeated on the surviving mice (arrow). FIG. 4a shows that the incubation with chicken annexin V (black triangles) clearly increases the tumor rejection in all groups in comparison to the injection with untreated, radiated tumor cells (white triangles).

To examine the effect on pre-established tumors 2.5×10⁴ living RMA tumor cells were injected subcutaneously into the left side of C57B1/6 mice. Each cohort contained 10 mice. Four days later either 1×10⁶ or 5×10⁶ radiated RMA tumor cells were injected into the right side. This injection was made after treatment of the radiated cells with (filled symbols) or without (unfilled symbols) chicken annexin V. FIG. 4b shows the time progression of the tumor-free animals. With the injection of 5×10⁶ tumor cells the treatment of radiated tumor cells with chicken annexin V significantly increases their immunogenicity and the thus resulting tumor rejection.

As shown in FIG. 5, the mice that were not completely protected had both elevated tumor latency (FIG. 5a) and survival time when the radiated tumor cells were treated with chicken annexin V before injection.

### C. DATA OF SCID MICE

### C1. GOAL

Determination of the effect of an AxV treatment of apoptotic cells on the innate unspecific immune system.

### C2. MATERIALS AND METHODS

### C2.1. CELLS

The human plasma cell line INA-6 was established from the pleura fluid of an eighty-year-old patient with plasma cell leukemia. It grows as single-cell suspension and shows the morphology of plasmablasts. The sub cell line INA-6 Tu1 originates from a passage in SCID mice. In contrast to the original cell line, INA-6 Tu1 proliferates regardless of interleukin-6 and forms tumors which can be felt significantly faster after intraperitoneal (i.p.) injection in SCID mice (Burger, R. et al. (2001) Hematol. J. 2: 42-53).

### C2.2. INDUCTION OF APOPTOSIS IN INA-6 Tu1 CELLS

The creation of apoptotic INA-6 Tu1 cells was performed by UV radiation for 40 seconds and then further cultivation for 12 hours. With this dose the cells show an apoptosis rate of approximately 90%, wherein an increase of the radiation dose only changed the rate insignificantly.

### C2.3. SCID MICE

Thirty female, six week old SCID mice (Charles River Wiga GmbH) were used for the experiment. Due to an innate defect, the SCID mice have no functional specific immune system in the form of mature T and B lymphocytes. The unspecific immune system of the animals is not affected, however. To eliminate any existing activity of natural killer cells (NK cells), the animals were radiated with a whole-body gamma radiation with an individual dose of 2 Gy.

### C2.4. INOCULATION WITH INA-6 Tu1

Twenty-four hours after the radiation the animals were each injected with 2×10⁷ vital (vitality rate of approximately 90%) INA-6 Tu1 cells in a 1 ml culture medium (RPMI 1640 with 20% fetal bovine serum) i.p..

### C2.5. THERAPEUTIC IMMUNIZATION

Twenty-four hours after the tumor inoculation the mice were divided into three groups of the same size. The animals of the control group each received an injection of 1 ml of Hanks' Balance Salt Solution (HBSS) i.p.. The animals of the second group each received an injection of 1 ml of BHSS with 2×10⁷ apoptotic INA-6 Tu1 cells i.p.. Then the animals of the third group each received an injection of 1 ml of HBSS with 2×10⁷ apoptotic INA-6 Tu1 cells, wherein the cells were incubated with 100 μg chicken annexin V at room temperature 30 minutes before the i.p. administration.

### C2.6. STATISTICAL ANALYSIS OF THE DATA

The statistical analysis of the data was performed by the Log-Rank test with the aid of software package SPSS 10.0.7 of SPSS Inc.

### C2.7. SUPPLEMENTAL EXPERIMENT

To be able to exclude a direct toxic effect of annexin on INA-6 Tu1 cells, a proliferation test was performed. For this 10⁴ INA-6 Tu1 cells were incubated for each little bowl in flat-bottom, micro-titer plates with 0.2 ml medium (RPMI-1640 with 10% fetal bovine serum and 2.5 ng/ml human recombinant interleukin-6) and different amounts of annexin V (6 pg/ml to 50 μg/ml) for 72 hours, wherein the cells were pulsed during the last 6 hours with 1 μCi [3H] thymidine (Amersham) per bowl. The cells were then harvested and analyzed in an β-scintillation counter. The values in FIG. 8 show the mean [³H] thymidine incorporation in cpm (counts per minute) from triplicates with the simple standard deviation as error bar. The curve clearly shows that annexin V has no effect whatsoever on the proliferation of INA-6 Tu1 cells.

### C3. OBSERVATIONS DURING THE ANIMAL STUDY

Forty-eight hours after the tumor inoculation the animals of the second group, but primarily the animals of the third group, showed strong, general signs of sickness such as unkempt fur and weight loss while the animals of the control group appeared outwardly healthy. On the third day and on the fifth day after the tumor inoculation one animal each of the third group died and one animal of the second group died on the 18th day after the tumor inoculation. The particular cause of death could not be determined. However, it can be excluded with considerable certainty that the animals died at this early time due to the induced myeloma illness. For this reason only the remaining nine or eight animals from the second and third groups were considered in the analysis of the experiment. Animals in the final stage of the illness were killed before the further examination.

### C4. RESULTS

On day 29 after the inoculation seven of the ten animals of the control group showed pea-sized tumors at the site of the inoculation. Comparable observations were also made in the second group where seven of nine animals showed clear tumor growth. Surprisingly no tumors could be determined at this time in any of the eight animals which were treated with annexin V-loaded apoptotic INA-6 Tu1 cells (group 3). The differences in data on tumor growth between group 3 and group 2 (p=0.0001) or the control group (p=0.004) are statistically significant (FIG. 9, table).

The mean survival duration after tumor inoculation was 54.8 days for the control group while the mice in the second group even showed a slightly shortened survival duration of an average of 50.4 days. In contrast the mean survival duration of six of the eight mice of the third group was clearly higher with 71.3 days. In addition the remaining two animals of the third group survived the previously specified end of the observation period (day 95) by nine days. Animals in which tumor appearance was delayed mainly showed liver infiltrates and formation of ascites while the growth of tumors which appeared early was mostly local. The differences in the survival data between group 3 and group 2 (p=0.0004) or the control group (p=0.0095) are statistically significant (FIG. 6, 7, table).

### C5. CONCLUSIONS

Participation of non-specific NK cells in addition to cytotoxic T cells directed to specific antigens in successful tumor resistance is general knowledge.

Due to a genetic defect, SCID mice have no specific immune systems and thus also have no functional cytotoxic T cells. However, they are able to suppress tumor growth with the help of their macrophages and NK cells via nitric oxide metabolites or/and perforine-caused cytotoxicity (Cifone, M. G., Ulisse, S., and Santoin, A. (2001) Int Immunopharmacol 1 (8): 1513-1524).

These data show that the therapeutic administration of annexin-loaded apoptotic cells can significantly delay the establishment of a human tumor in SCID mice and thus lengthen the length of survival.

Apparently apoptotic cells are changed by the annexin load so that they are able to activate the functioning unspecific immune system—in particular NK cells and macrophages—of SCID mice.

Primarily the upward regulation of the monokine TNF-alpha due to the annexin-loaded apoptotic cells (FIGS. 6, 7) could be responsible here for the activation of the NK cells. Because TNF-alpha is particularly important for the recruiting of NK cells in the peritoneum since TNF-alpha-deficient mice show a deficiency here (Smyth, M. J. et al. (1998) J Exp Med 9: 1611-1619).

1-33. (Canceled)

34. A composition for stimulating the innate non-specific immune system, comprising:
a polypeptide selected from the group consisting of a) an annexin V polypeptide having an amino acid sequence shown in SEQ ID NO: 1 or 2; b) a polypeptide having an amino acid sequence at least 80% identical to SEQ ID NO: 1 or 2; and c) an immunogenic fragment of a) or b).

35. The composition of claim 34, wherein said polypeptide of b) has an amino acid sequence at least 85% identical to SEQ ID NO: 1 or 2.

36. The composition of claim 34, wherein said polypeptide of b) has an amino acid sequence at least 90% identical to SEQ ID NO: 1 or 2.

37. The composition of claim 34, wherein said polypeptide of b) has an amino acid sequence at least 95% identical to SEQ ID NO: 1 or 2.

38. The composition of claim 34, wherein the polypeptide binds to phosphatidylserine.

39. The composition of claim 34, wherein the annexin V polypeptide is not a human annexin V.

40. The composition of claim 39, wherein the annexin V polypeptide is a chicken annexin V polypeptide.

41. The composition of claim 34, further comprising tumor cells.

42. The composition of claim 41, wherein the tumor cells are human tumor cells.

43. The composition of claim 42, wherein said human tumor cells are apoptotic and/or necrotic tumor cells.

44. The composition of claims 42, wherein the human tumor cells are in contact with the arinexin V polypeptide.

45. A method for producing a composition for stimulating the innate non-specific immune system, comprising: contacting apoptotic and/or necrotic tumor cells with a polypeptide selected from the group consisting of a) an annexin V polypeptide having an amino acid sequence shown in SEQ ID NO: 1 or 2; b) a polypeptide having an amino acid sequence at least 80% identical to SEQ ID NO: 1 or 2; and c) an immunogenic fragment of a) or b).

46. The method as defined in claim 45, wherein the polypeptide or fragment thereof binds to phosphatidylserine.

47. The method of claims 45, wherein the annexin V polypeptide is not a human annexin V polypeptide.

48. The method of claim 45, wherein the annexin V polypeptide is a chicken annexin V polypeptide.

49. The method of claim 45, wherein said apoptotic and/or necrotic tumor cells are in vivo.

50. The method of claim 45, wherein apoptosis and/or necrosis in the apoptotic and/or necrotic tumor cells, respectively, is triggered by radiation of the tumor cells ex vivo or in vivo.

51. The method of claim 45, wherein apoptosis and/or necrosis in the apoptotic and/or necrotic tumor cells, respectively, is triggered by bringing the tumor cells into contact with cytostatic drugs.

52. The method of claim 45, wherein the polypeptide is obtained by expression in transformed strains of Escherichia coli and then purified.

53. The method of claim 52, wherein the strain of Escherichia coli is Escherichia coli BL21 (DE3).

54. The method of claim 52, wherein pDJ2-AnXV is used to express the polypeptide.

55. The method of claim 52, wherein the purification of the expressed polypeptide is by column chromatography.

56. The method of claim 55, wherein the column chromatography uses a salt gradient.

57. A method of treating or vaccinating an individual, comprising: administering an effective amount of a composition to the individual, wherein the composition comprises a polypeptide selected from the group consisting of a) an annexin V polypeptide having an amino acid sequence shown in SEQ ID NO: 1 or 2; b) a polypeptide having an amino acid sequence at least 80% identical to SEQ ID NO: 1 or 2; and c) an immunogenic fragment of a) or b), wherein the effective amount is an amount effective to stimulate the innate non-specific immune system.

58. The method of claim 57, wherein the composition further comprises tumor cells.

59. The method of claim 58, wherein the tumor cells are human tumor cells.

60. The method of claim 59, wherein the human tumor cells are apoptotic and/or necrotic tumor cells.

61. The method of claim 57, wherein the treatment is for combating bacteria, viruses, parasites, or tumors.

62. The method of claim 61, wherein the virus is a retrovirus, a lentivirus, sindisvirus, or HIV.

63. The method of claim 61, wherein the parasite is Trypanosoma brucei or a Plasmodium spp.

64. The method of claim 61, wherein the bacteria is Treponema pallidum.

65. The method of claim 57, wherein the vaccination is a tumor vaccine or a malaria vaccine.
